(19) United States
(12) Patent Application Publication
(10) Pub. No.: US 2005/0137542 A1
(43) Pub. Date: Jun. 23, 2005

(54) Live graphics on absorbent articles using electrochromic displays

(75) Inventors: Rick Underhill, Neenah, WI (US); Jeffrey Dean Lindsay, Appleton, WI (US); Fung-jou Chen, Appleton, WI (US); Gil Bernard Didier Garnier, Neenah, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/742,611

(22) Filed: Dec. 19, 2003

(51) Int. Cl. A61F 13/15; A61F 13/20

(52) U.S. Cl. 604/361

(57) ABSTRACT

An absorbent article is provided that includes an electroactive display. The electroactive display may include an electrochromic composition positioned between two electrodes. The electroactive display is configured to create and exhibit an image that contains active features such as moving graphics or color-changing objects. For instance, the electroactive display may display an animated cartoon character or a moving vehicle. The electroactive display may also create symbols or words. When combined with sensors, a power source, and the proper circuitry, the electroactive display may be used, for instance, as a wetness indicator or to indicate the presence of a particular substance. In other embodiments, the electroactive display may be used solely for aesthetic purposes.

### BACKGROUND OF THE INVENTION

Almost all incontinence products sold today including diapers, training pants, adult incontinence products, absorbent swimwear, and the like are manufactured to be disposed of after a single use. The absorbent articles typically contain a cover material, a liner, and an absorbent structure positioned between the cover material and the liner. The absorbent structure can be made of, for instance, superabsorbent particles. Many absorbent articles, especially those sold under the trade name HUGGIES™ by the Kimberly-Clark Corporation, are so efficient at absorbent liquids that it is sometimes difficult to tell whether or not the absorbent article has been insulted with a body fluid.

Accordingly, various types of moisture or wetness indicators have been suggested for use in absorbent articles. The wetness indicators may include alarm devices that are designed to assist parents or attendants identify a wet diaper condition early on. The devices produce either a visual or an audible signal.

In the past, the visual wetness indicators have typically comprised simple color changing materials. As such, a need currently exists for a more sophisticated wetness indicator. In particular, a need exists for a wetness indicator that not only indicates wetness to a parent or attendant but can also be used as an actual training tool that may motivate a child to become potty trained.

A need also exists for a display device that may be used in conjunction with absorbent articles and not necessarily solely for the purpose of indicating wetness. For instance, a need also exists for absorbent articles being equipped with a display device that, instead of indicating wetness, may indicate other conditions. For instance, a display device is needed that may be used in adult incontinence products for indicating the presence of an analyte in a body fluid. A need also exists for a display device that may be used simply to improve the aesthetic appeal of an absorbent article.

### SUMMARY OF THE INVENTION

The present invention is directed generally to an electroactive display that may be incorporated into an absorbent article. The absorbent article may be, for instance, a diaper, training pant, incontinence product, medical garment, bandage, absorbent swimwear, feminine hygiene product, and the like. The electroactive display in accordance with the present invention comprises an electrochromic composition positioned between a first electrode and a second electrode. By feeding an electrical current to the electroactive display, the electroactive display is configured to create an image containing an active feature, such as moving features and multiple colors. As used herein, a “moving feature” refers to a feature in the image that appears and disappears over time and/or refers to a feature in the image that traverses across the display panel in some manner. More generally, an “active feature” can include not only moving features, but also objects or portions of objects that change color. Active features can also involve changes in the shape and size of objects, changes in displayed text or graphical symbols conveying information, including graphical depiction of sensor data, and changes in the intensity of a displayed color (e.g., brightness, luminosity, or saturation).

Of particular advantage for some embodiments, the electroactive display can be economically produced, may have a thickness of less than about 5 millimeters or less than about 3 millimeters, can be flexible, and can be easily incorporated into any suitable absorbent product.

Once attached to a power supply, the electroactive display can be manufactured to produce numerous graphics depending upon the particular circumstances. For instance, for diapers and training pants, the electroactive display may be configured to produce moving cartoon characters, moving vehicles, live graphic fanciful landscape scenes, and the like. In other embodiments, the electroactive display may be configured to produce symbols, words and phrases to indicate a particular condition. In still other embodiments, the electroactive display may be configured to produce appearing and disappearing faces, toys, landscapes, vehicles, animals, and the like.

In one embodiment, the electroactive display can be customized, such that custom graphics in the form of a digital image file may be downloaded from a computer, PDA, digital camera, video-equipped cellphone, and the like, into a memory component associated with the electroactive display device, such that a representation of the digital image can be displayed by the electroactive display device. The image file may comprise two or more images that can be alternately displayed to provide active graphics, or may comprise a single image that appears and disappears, or may comprise a static image that serves as a background upon which active graphics elements such as cartoon characters may be displayed. In such applications, the image may be processed to a form compatible with the color display capabilities of the electroactive display device. This processing may be done by a user using any known image editing software (e.g., PHOTOSHOP© by Adobe Systems of San Jose, Calif., or DRAWPLUS 7 by Serif Ltd. of Nottingham, England), by dedicated processing software marketed for use with the electroactive display technology, or by software associated with the memory component or the electroactive display device. For example, a user may select a series of 10 24-bit color images and apply software tools to convert the images into an animated GIF file that can be viewed using a Web browser or other software on a computer. The animated GIF file displays the series of images with embedded instructions controlling the display time of each image and how one image replaces the previous image. This GIF file may contain many more colors than can be displayed with a given electroactive display device, or may contain much higher resolution (pixel count) than can be displayed on the electroactive device.

To adapt the animated GIF for electroactive display, converting software may provide the user with the option to reduce the color count to an appropriate level (e.g., two, three, or four colors, including a black and white image), optionally with dithering, smoothing, or other steps to improve the display of the converted image. The display time of each image in the animated series may also be manually or automatically adjusted to improve compatibility with the refresh rate or other capabilities of the electroactive display device. In this manner, for example, a parent may be able to download a series of images of a child's favorite pet or toy that can be displayed in animated form on an electroactive display device associated with a product, which can be a wearable product, a disposable product, a toy, product packaging, or another object. Downloading of the converted image for use by the electroactive display device may be done by wireless transmission (e.g., the memory component may be memory in a thin writable RFID device), by a inserting chip or memory card loaded with the image by prior transfer from a computer into a suitable receptacle associated with the electroactive display, by a cable connection such as USB2, and the like.

In general, any suitable electrochromic composition may be used in the electroactive display of the present invention that is capable of receiving a current for forming an image having active features (e.g., moving objects, appearing and disappearing objects, objects or portions thereof that change colors, shape, or size, etc.). For instance, in one embodiment, the electrochromic composition may be present between the electrodes in the form of pixels that are selectively activated, for instance, by a controller. In one particular embodiment, the electrochromic composition may comprise an electrochromic compound that undergoes a reversible electron transfer reaction resulting in a pH gradient. The electrochromic compound, for instance, may undergo reversible oxidation/reduction reactions when exposed to a certain amount of voltage. The electrochromic composition may further include a pH indicator and a conductive material for transporting ions from the electrochromic compound to the pH indicator. When used in conjunction with a pH indicator, the electrochromic compound may be bistable, meaning that the compound can remain in multiple color states without the need for persistent voltage to be applied. For example, a bistable electrochromic compound turns a different color under application of a voltage and will remain a changed color after the voltage is removed.

The electrodes positioned in communication with the electrochromic composition may be any suitable conductive films capable of serving as an anode and a cathode. For most applications, at least one of the electrodes may be transparent. The transparent electrode may be, for instance, a film containing a metal doped metal oxide. For instance, in one embodiment, the transparent electrode may comprise a polyester film containing indium tin oxide.

In order for the electroactive display to create and form an image, the display is connected to a power supply. The power supply may be, for instance, a battery, including a flexible thin-film battery or printed battery comprising flexible materials. The battery may be, in one embodiment, an electrochemical cell. In another embodiment, the battery may comprise a radio frequency identification device that generates power when sensing an RF source.

When the battery is an electrochemical cell, the battery may be formed by printing electrodes onto substrates to form a thin device having a thickness, for instance, of less than about 3 mm, such as less than 2 mm, and even less than 1 mm. These types of electrochemical cells are particularly advantageous since they may be easily incorporated into an absorbent article. In one embodiment, for instance, the electrochemical cell may contain a zinc anode and a manganese dioxide cathode.

The electroactive display of the present invention may be used for numerous and wide varying purposes when incorporated into an absorbent article. For instance, in one embodiment, the electroactive display may be used simply in order to improve the aesthetic appeal of the product. In another embodiment, the electroactive display may be used on a diaper or training pant to indicate wetness. In this embodiment, the electroactive display may be connected to a sensor, such as a moisture sensor, a conductivity sensor or a temperature sensor. Once activated, the sensor may cause the electroactive display to undergo a change to indicate that the absorbent article has been insulted with a body fluid. For instance, in one embodiment, once activated, the sensor may cause the electroactive display to shutdown. In another embodiment, the sensor may cause the electroactive display to display a symbol or word indicating the need to change the article. In one embodiment, the sensor may cause the electroactive display to undergo a change that may be readily discerned by a toddler in a manner that may motivate the toddler to become potty trained.

In other embodiments, the electroactive display may be connected to a biosensor for use, for instance, in adult incontinence products and feminine hygiene products. The biosensor may be activated when contacted with an analyte contained in a body fluid. The analyte, for instance, may be a protein, glycoprotein, antibody, antigen, hemoglobin, enzyme, metal salt, hormone, and the like. In this embodiment, for instance, the electroactive display may be used to indicate a possible health problem that exists with the wearer. For instance, in one particular embodiment, the biosensor may be used to indicate the presence of a protein for indicating a possible kidney problem.

In still another embodiment, the electroactive display may be combined with a timing circuit. The timing circuit can be configured to cause the image to change in a predetermined manner after a preselected period of time. For example, in this embodiment, the timing circuit, after the preselected period of time, may indicate that a particular action is needed. For example, if incorporated into absorbent swimwear, the electroactive display may be used to indicate the need to reapply sunscreen. In other embodiments, the combination of a timing circuit and electroactive display may be used to indicate the need to replace the disposable absorbent article if the article, for instance, is a diaper or a bandage.

The electroactive display may be flexible for improved comfort in an absorbent article. For example, a display panel having a display area of about 10 square centimeters or greater, about 50 square centimeters or greater, about 100 square centimeters or greater, or about 200 square centimeters or greater, may have sufficient flexibility such that any portion from all or a majority of the display area at 25° C. can be deformed to bend around a rigid rod having a fixed diameter of 1 inch (2.54 centimeters) without causing fracture or failure of the display panel. An electroactive display capable of passing this test can be said to have a Flexibility Diameter of 1-inch or less. If an article can be safely deformed using the aforementioned procedure but with a 2-inch diameter rod instead of a 1-inch diameter rod, then the display panel may be said to have a Flexibility Diameter of 2 inches or less. The electroactive display panels of the present invention may have a Flexibility Diameter of about 2 inches or less, about 1 inch or less, or about 0.5 inches or less.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1A is a perspective view of one embodiment of an absorbent article made in accordance with the present invention including an electroactive display;

FIGS. 1B, 1C and 1D are partial perspective views of the embodiment shown in FIG. 1A illustrating various moving features on the electroactive display;

FIG. 2 is an exploded perspective view showing one embodiment of an electroactive display made in accordance with the present invention;

FIG. 3 is an exploded perspective view of another embodiment of an electroactive display made in accordance with the present invention; and

FIG. 4 is another embodiment of an absorbent article made in accordance with the present invention.

Repeat use of reference characters in the present specification and drawings is intended to indicate the same or analogous features or elements of the invention.

### DETAILED DESCRIPTION OF THE INVENTION

It is to be understood by one of ordinary skill in the art that the present discussion is a description of exemplary embodiments only, and is not intended as limiting the broader aspects of the present invention.

According to the present invention, there is provided an electroactive display that is incorporated into a disposable absorbent article. The absorbent article may be, for instance, a diaper, a training pant, an incontinence product, a feminine hygiene product, a medical garment, a bandage, absorbent swimwear, and the like. The term “disposable” refers to articles which are designed to be discarded after a limited use rather than being laundered or otherwise restored for reuse. The electroactive display may be used for numerous and various purposes. For instance, in one embodiment, the device may be used as a wetness indicator. Unlike previous diaper wetness detectors, however, the electroactive display is capable of producing an image that contains moving features. For instance, the image may contain multiple colors and can include animated cartoon characters, moving vehicles, and fanciful landscape depictions in which elements appear, disappear, and reappear over time. In fact, instead of or in addition to being used as a wetness indicator, the electroactive display may actually be used to motivate a child to become potty trained.

It should be understood, however, that in addition to being used as a wetness indicator, the electroactive display may be used for various and numerous other reasons. For example, the electroactive display may be incorporated into an absorbent article solely for the purpose of improving the aesthetic appeal of the article.

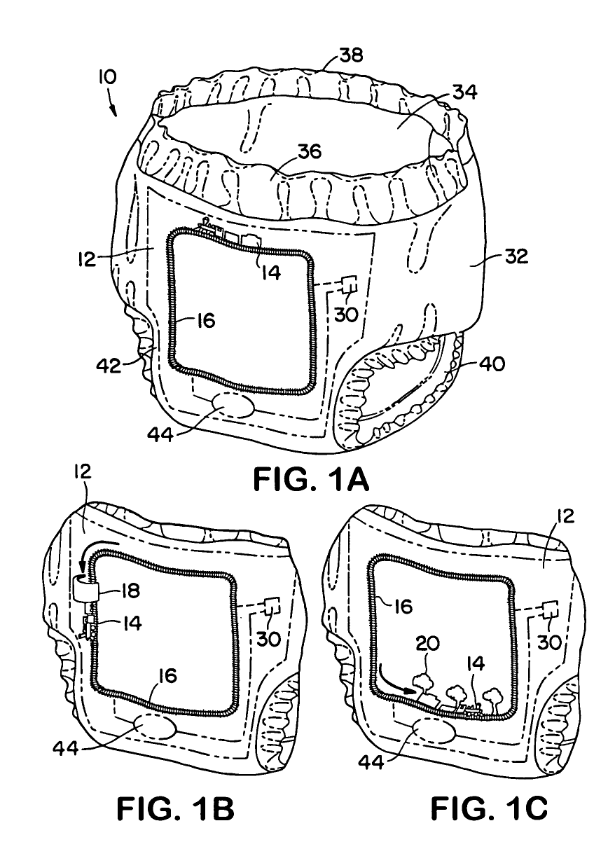

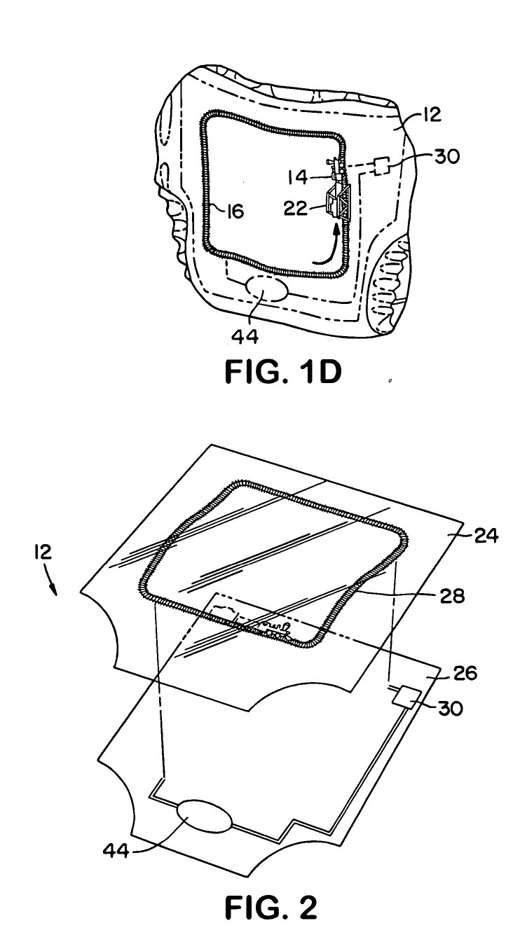

Referring to FIG. 1A, one embodiment of an absorbent article made in accordance with the present invention is shown. For purposes of explanation, in this embodiment, the absorbent article represents a diaper 10. As shown, the diaper 10 includes an electroactive display 12 in accordance with the present invention. The electroactive display generally comprises an electrochromic composition positioned between a first electrode and a second electrode. When voltage is applied to the display, the electrochromic composition undergoes a reversible color change. If controlled appropriately, the electrochromic composition can form and create an image on the absorbent article.

For instance, FIGS. 1A-1D are intended to represent how an image created by the electroactive display 12 may change over time. In this particular embodiment, a train 14 is illustrated traveling on a track 16 that forms a loop on the front of the diaper 10. While the train 14 is traveling on the track 16, various features can appear, disappear, and reappear in a fanciful manner. For example, in FIG. 1B, the train 14 is shown traveling through a tunnel 18. The tunnel 18 may always appear in the image or appear in the image as the train approaches. In FIG. 1C, the train is passing by a group of trees 20, while in FIG. 1D, the train is shown crossing over a bridge 22.

It should be understood, however, that in addition to a moving vehicle, various other animated images may appear on the electroactive display 12. In other embodiments, an animated character may be shown engaging in some type of activity. For instance, the animated character may move across the display and, for instance, greet other animated characters. Alternatively, the characters may smile, wink, wave, blush, and the like. They or other objects in the display may also be associated with one or more sound generation devices capable of playing a sound such as music, a prerecorded voice, sound effects, and the like, that may be synchronized with the visual display to provide audio signals corresponding to animated displays. For example, the sound of a train whistle may be periodically played as an animated train appears to move on the display panel.

Referring to FIG. 2, an exploded view of the electroactive display 12 is shown. In general, the electroactive display 12 includes a first electrode 24, a second electrode 26, and an electrochromic composition 28 positioned between the first electrode 24 and the second electrode 26. In general, any suitable electrochromic composition may be used in the electroactive display as long as the composition is capable of being manipulated in a way to show moving features. For example, electrochromic compositions and devices are disclosed in PCT International Publication No. WO 02/075441, PCT International Publication No. WO 98/35267, PCT International Publication No. WO 01/27690, PCT International Publication No. WO 97/35227, U.S. Pat. No. 5,796,345, and PCT International Publication No. WO 03/48998, which are all incorporated herein by reference to the extent they are not contradictory. An electronic display system is also commercially available under the trade name COMMOTION™ by Dow Global Technologies, Inc., which is believed also to be usable in the present invention.

As shown in FIG. 2, the electrochromic composition is positioned between the first electrode 24 and the second electrode 26. The electrochromic composition is positioned between the two conductive electrodes in order for a voltage to be applied across the display elements. The electrodes 24 and 26 can be made from any suitable material. The first electrode 24, however, should also be transparent or substantially translucent.

In the illustrated embodiment, the electroactive display only includes one set of electrodes. It should be understood, however, that the electroactive display may include a plurality of electrode sets including multiple anodes and multiple cathodes. The electrode pairs may be present in series or as separate circuits.

As shown in FIG. 2, the electrodes 24 and 26 can be in the form of a film. Examples of suitable electrode materials include a polymer film containing a metal doped metal oxide. For instance, in one embodiment, the electrode may be a polyester film (made from polyethylene terephthalate) coated with indium doped tin oxide. Such films are commercially available from Vision Tek Systems Ltd. of England. ITO/PET films are generally produced by sputtering indium tin oxide on a PET layer in a controlled vacuum environment, optionally with laser or chemical etching. Electron beam processing is also possible. Further, in other embodiments, the metal doped metal oxide may be printed directly onto the film.

In other embodiments, the one or more electrodes may be formed from a modified porous nanocrystalline film. Such films are believed to be commercially available from NTera Devices. In this embodiment, the film may comprise a nanostructured metal oxide film, doped to metallic levels of conductivity. Still further examples of electrodes include other transparent conductive metal oxides, as well as thin transparent films of metals, such as gold. A conducting polymer film may also be used as an electrode.

The electrochromic composition positioned between the electrodes comprises a material that is capable of changing color when exposed to an electric potential. Most electrochromic compositions, for instance, undergo oxidation/reduction reversible reactions when exposed to a voltage. For instance, a material may be one color in its oxidized form and another color in its reduced form.

In one particular embodiment of the present invention, the electrochromic composition includes an electrochromic compound that undergoes a reversible electron transfer reaction with a subsequent change in its protic state resulting in a pH gradient in the device. The electrochromic compound may be combined with a pH indicator and a charge transport material that transports ions from the electrochromic compound to the pH indicator.

The actual electrochromic compound used may depend upon the particular application. In general, the electrochromic compound should preferentially undergo a redox reaction relative to the other components. For instance, the standard reduction potential of the electrochromic compound should be less for the other components in the device.

Examples of compounds suitable for use as the electrochromic compound may include but are not limited to any number of organic or inorganic redox reagents, including but not limited to: iodates, bromates, sulfates, metal hydroxides, phosphates, ketones, aldehydes, quinones, quinolines, sulfur compounds, hydroxybenzenes, carboxylic acids, polyoxometallates, and amines. Materials such as hydroquinone and other quinone derivatives such as methylquinone and duroquinone, which are highly reversible, do not undergo many side reactions, and have a relatively low standard reduction potential may be desirable in certain applications. The electrochromic compound is preferably present in amounts of greater than 0.01 percent, more preferably greater than 0.1 percent based on total weight of the composition. The electrochromic compound may be present in amounts less than about 15 percent, such as less than about 10 percent, based on total weight of the composition.

The electrochromic compound may also be combined with a secondary electrochromic material which may undergo a complimentary redox reaction. A complimentary redox reaction is defined as the material which undergoes the second half of the redox reaction (i.e. one of the preferential half reactions at the electrode surface). Furthermore, the secondary electrochromic compound should be reversible (electrochemically) and chemically stable in the system. Examples of compounds suitable for use as the secondary electrochromic compound may include but are not limited to any number of organic or inorganic redox reagents, including but not limited to: iodates, bromates, sulfates, metal hydroxides, phosphates, ketones, aldehydes, quinones, quinolines, sulfur compounds, hydroxybenzenes, carboxylic acids, polyoxometallates, and amines. Materials such as hydroquinone and other quinone derivatives such as methylquinone and duroquinone, are highly reversible, do not undergo many side reactions, and have a relatively low standard reduction potential. When used, the secondary electrochromic compound should be present at concentration ranges equal to those used in the electrochromic compound and at ratios optimized for the individual cell (i.e. electrochemical system). Thus, the secondary electrochromic compound may be present in amounts of greater than 0.01 percent, such as greater than 0.1 percent based on total weight of the composition. The secondary electrochromic compound may be present in amounts less than about 15 percent, such as less than about 10 percent, based on total weight of the composition.

As described above, the electrochromic compound causes a pH gradient. In this embodiment of the present invention, the composition thus contains a pH indicator. Any known pH indicator dyes or their derivatives may be used. A single indicator dye may be used or they may be used in combination to give a variety of colors. The response and chromaticity of various dyes can be optimized by changing the starting pH of the system and/or the proton or hydroxide generator. Non-limiting examples of suitable indicator dyes include phenolphthalein, bromocrescol purple, phenol red, ethyl red, quinaldine red, thymolthalein, thymol blue, malachite green, crystal violet, methyl violet 2B, xylenol blue, cresol red, phyloxine B, congo red, methyl orange, bromochlorophenol blue, alizarin red, chlorophenol red, 4-nitrophenol, nile blue A, aniline blue, indigo carmine, bromothymol blue, etc. Dyes that yield more than two different colors, depending on pH, are of particular interest as they would enable multi-color images with use of a single dye. Thymol blue is one example of such a dye—it is yellow under neutral conditions, red under acidic conditions, and blue under basic conditions. Dyes that are very pale or transparent in one form are also desirable as they may allow more flexibility in color selection in the display. Finally, indicator dyes, which change colors at varying pH levels and are of varying colors, may be combined to tailor the colors in the display to the users desire or to attain multi-color or possibly full color displays. The indicator dye is present in amounts of at least 0.01 percent, such as 0.1 percent by weight. The dye may be used in amounts less than 15 weight percent, such as less than 5 weight percent. When combinations of dyes are used, the total amount of dye in the composition may be less than 15 percent. Other non pH sensitive dyes or pigments may be used to alter the aesthetics of the display as well, as long as the materials do not parasitically alter the redox chemistry, such that the system can no longer meet the application requirements.

In one embodiment, the electroactive display may comprise multiple layers of electroactive materials in a superposed relationship to provide overlapping colors or multiple pixel colors near one another to provide a variety of apparent colors when viewed from a distance such as from about 20 centimeters or 100 centimeters or greater.

The conductive material for transporting ions may be any known material that is capable of transporting the necessary ions from the redox material to the indicator dye.

Examples of materials which can be used as the conductive transport material include aqueous solutions, protic solvents, and solid electrolytes. The aqueous solutions may comprise electrolyte concentrations of greater than or equal to 0.01 percent and less than or equal to 50 percent, such as less than or equal to 0.5 percent based on weight of the solution. Suitable electrolyte components include salts, such as, for example, sodium, lithium, magnesium, or calcium sulfate, percholorate or chloride, as well as organic ionic materials, such as amines and organic acid electrolytes. Non-chloride electrolytes are desirable in some applications because chloride is fairly reactive with metal electrode surfaces. The presence of a high concentration of other ions utilizes the common ion effect to reduce the neutralization driving force of the protons and hydroxide ions, thus enhancing open circuit lifetime.

Optionally, the electrolyte solution may contain one or more buffer components depending on the operating pH range of the system. A buffer is defined as a material that resists changes in pH, as a result of addition of small amounts of acids or bases. By adding the appropriate pH buffer(s) to the conductive transport material, lifetimes may be enhanced by avoiding pH extremes at the electrodes. Examples of buffer components include, but are not limited to: weak acids such as carboxylic acids (formate, acetate, citrate, fumaric, glycolic, oxalic, etc.), weak bases such as amines (ethylenediamine, triethylamine, etc.), or zwitterionic materials such as amino acids or biological buffers (CAPS, MES, MOPS, TAPSO, or AMPSO).

The composition may also comprise a co-solvent. The co-solvent may be useful to enhance component solubility, modify conductivity, modify rheology of the composition and modify adhesion to the surface of the electrode layer. Potentially useful co-solvents include, but are not limited to: alcohols such as isopropanol and ethanol, aldehydes, ketones, ethers, formamides, or common electrochemical solvents such as acetonitrile, N-methylpyrolidinone, and proplyene carbonate.

A nonaqueous system may be used as the conductive transport material, provided the redox component can cause an adequate pH shift and there is adequate polarity to provide good ionic conductivity. Preferably, these systems comprise an electrolyte component as specified for example above, in a protic solvent. Suitable protic solvents that could be used in a non-aqueous system include, but are not limited to: propylene carbonate,dimethyl formamide, N-methyl pyrrolidinone, acetonitrile, dimethylsulfozide, alcohols (methanol, isopropanol, ethanol, etc.), pyridine, and 1,4-dioxane. In addition, a low molecular weight glycol ether such as ethylene glycol, propylene glycol, polyethylene glycol, or a derivative therefore may be used.

A solid system may also be used as the charge transport material. Examples of such systems include conductive polymers such as polyacrylamidomethyl-propanesulfonate (POLYAMPS), polystyrene sulfonic acid (PSSA), and copolymers, and blends and block copolymers thereof. The conductive polymer may be used alone (i.e., a solid electrolyte system) or in a swollen aqueous or solvent solution. In addition, the electrolyte material may be physically separated from the redox materials and/or the indicator dye, whereby the dye and/or redox active species are placed on either side of the electrolyte in a “battery-like” structure. The materials could be applied by any number of manufacturing processes, including but not limited to printing processes such as silk-screening, ink jetting, roll printing, or stenciling.

The amount of ion/charge transport material in the system may depend upon the efficiency of the material in transporting charge and/or ions, as well as the relative amounts of additional additives. However, the amount is typically at least 5, such as at least 10, or at least 20 weight percent and is less than 99.98 weight percent, such as less than 70 weight percent.

The composition may also comprise a matrix material. The matrix material may provide structural integrity to the device. This will aid printability and processability. In addition, or alternatively, the matrix material may be used to control ion transport, and diffusion rate of the other materials in the composition. Limiting ion transport and diffusion of components in the longitudinal direction increases resolution and stability over time of the image formed. Limiting ion transport and diffusion in all directions increases open circuit lifetime and optical density. Thus, according to one embodiment, the matrix material may comprise a skeletal, porous or framework structure that is saturated with the other components of the composition. For example, an open cell polymeric foam, a honeycomb structure, a screen, a mesh, spacer particles or paper may be saturated with the other components or have the other components absorbed into the open regions of the structure. Naturally and synthetically occurring polymers are particularly suitable for supplying such skeletal or porous structures. Alternatively, or in addition to a skeletal matrix material, viscosity modifier or diffusion inhibitor may be blended directly with the other components. Polymers and other viscosity modifiers may be used. Multiple matrix materials may also be added. For example, fumed silica is known to disrupt the crystalinity of glycol ethers, thus increasing the conductivity of the system while maintaining good structural integrity. Precise choice of such a matrix material will depend upon compatibility with the solution or solvents that are chosen. Nanocrystalline particles or sol gel systems may also be added as well to optimize the rheological properties of the system while maintaining the required transport properties. Examples of matrix materials include silicates such as silicon dioxide, aluminates, or zirconium oxide, barium titanate and other particles or polymeric materials such as, hydroxyethyl cellulose, polyethylene glycols, polyethylene oxides, polyurethanes, polyacrylates, polysulfonic acids, polyacetates, latexes, styrene divinylbenzene polymers, and polypropylenes.

The electroactive device may be assembled using known methods, such as vapor deposition, electroplating, etc. In one embodiment, the electrochromic composition is printed, such as screen printed onto the electrodes. In one embodiment, the electrochromic composition is present between the electrodes in the form of pixels that are selectively activated. For instance, each pixel may be activated independently or simultaneously using a drive chip. In order to form moving features, each pixel may contain individual transistors, with the opposite electrode serving as the ground electrode. The transistors may be individually patterned, deposited or printed. Each pixel may be adapted to display an electronically determined color. The pixel may be able to switch between two colors (e.g., colorless or white versus a color such as red or yellow, or may be able to switch between two non-white colors such as red and blue, yellow and green, blue and pink, and the like), or between three colors, four colors, or any number of colors. Different types of pixels each capable of switching between two or more colors may be distributed in a pattern to allow differing colors to be displayed in an adjoining relationship to create the appearance of new colors, similar to the use of distributed red, green, and blue pixels in a television screen. When pixels are used the resolution in one or more directions may be any number of pixels per centimeter, such as about 1 pixel per centimeter or greater, about 5 pixels per centimeter or greater, about 10 pixels per centimeter or greater, about 20 pixels per centimeter or greater, and the like.

In order for the electroactive display to produce an image, the electroactive display 12 is connected to a power supply 30, such as a battery as shown in FIGS. 1A-1D and FIG. 2. The battery 30 may be directly incorporated into the electroactive display 12 or may be connected to the electroactive display through conductive leads. In general, any suitable battery may be used in conjunction with the electroactive display. The battery 30, however, should be capable of being easily incorporated into the diaper 10 and may be thin and flexible.

In one embodiment, the battery 30 may be an electrochemical cell. For example, a suitable electrochemical cell is marketed currently by Power Paper Ltd., which is located in Israel.

Suitable batteries are disclosed, for instance, in PCT International Publication Number WO 00/62365, PCT International Publication Number WO 03/017392, PCT International Publication Number WO 03/035167, PCT International Publication Number WO 03/035166, PCT International Publication Number WO 02/102273, PCT International Publication Number WO 01/58506, PCT International Publication Number WO 00/62248, PCT International Publication Number, WO 98/56458, U.S. Pat. No. 5,897,522, and U.S. Pat. No. 6,284,352, which are all incorporated herein by reference.

For instance, in one embodiment, the electrochemical cell includes an anode layer, a cathode layer, and a layer of an electrolyte. The electrolyte layer typically includes a porous insoluble substance, such as filter paper, a plastic membrane, a cellulose membrane, cloth, and the like. In one embodiment, the electrochemical cell includes a zinc anode and a manganese dioxide cathode. The anode and cathode can be printed onto a substrate.

The electrochemical cell can produce an amount of power sufficient to cause changes in the electroactive display 12. For instance, the electrochemical cell may have a nominal voltage of at least 1.0 volts, such as at least 1.5 volts. The thickness of the electrochemical cell may be less than about 3 mm, such as less than about 2 mm. For instance, in one embodiment, the electrochemical cell may have a thickness of less than about 1 mm, such as less than about 0.08 mm.

The electroactive display 12 and the battery 30 may be incorporated into the diaper 10 in any suitable manner. In one embodiment, for instance, the electroactive display 12 may be adhered to an outer cover 32 of the diaper 10 using a suitable adhesive. In one embodiment, a window may be cut out of the outer cover 32 and the electroactive display 12 may be positioned behind the window. For instance, the edges of the window may overlap with the edges of the electroactive display 12.

In an alternative embodiment, the electroactive display may be designed to be removed from the diaper 10 and reused on other absorbent articles. In this embodiment, for instance, the electroactive display 12 may be attached to the diaper using a removable attachment device, such as a pressure sensitive adhesive, hook and loop fasteners, and the like. Alternatively, the electroactive display 12 may be integral with or fixedly attached to the article to prevent removal.

In general, many absorbent articles made in accordance with the present invention including the diaper 10 as shown in FIG. 1A include an outer cover 32, an inner lining 34, and an absorbent structure (not shown) positioned between the outer cover and the inner lining. As shown in FIG. 1A, the diaper 10 may also include elastic waistbands 36 and 38 and elastic leg members 40 and 42.

The diaper 10 as shown in FIG. 1A can be made from various materials. The outer cover 32 may be made from a material that is substantially liquid and permeable, and can be elastic, stretchable or nonstretchable. The outer cover 32 can be a single layer of liquid and permeable material, or may include a multi-layered laminate structure in which at least one of the layers is liquid and permeable. For instance, the outer cover 32 can include a liquid permeable outer layer and a liquid and permeable inner layer that are suitably joined together by a laminate adhesive.

For example, in one embodiment, the liquid permeable outer layer may be a spunbond polypropylene nonwoven web. The spunbond web may have, for instance, a basis weight of from about 15 gsm to about 25 gsm.

The inner layer, on the other hand, can be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm.

A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent structure is positioned in between the outer cover and a liquid permeable bodyside liner 34. The bodyside liner 34 is suitably compliant, soft feeling, and non-irritating to the wearer's skin. The bodyside liner 34 can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 34. For example, the bodyside liner can be made from a meltblown or spunbonded web of polyolefin fibers. The bodyside liner can also be a bonded-carded web composed of natural and/or synthetic fibers.

A suitable liquid permeable bodyside liner 34 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

The material used to form the absorbent structure, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.1 to about 0.45 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a suberabsorbent material is capable of absorbing at least about 15 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure.

The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. Nos. 4,100,324 to Anderson, et al.; 5,284,703 to Everhart, et al.; and 5,350,624 to Georger, et al.; which are incorporated herein in their entirety by reference for all purposes.

It is also contemplated that elastomeric absorbent web structures may be used. For example, an elastomeric coform absorbent structure having from about 35% to about 65% by weight of a wettable staple fiber, and greater than about 35% to about 65% by weight of an elastomeric thermoplastic fiber may be used to define absorbent pad structures according to the invention. Examples of such elastomeric coform materials are provided in U.S. Pat. No. 5,645,542, incorporated herein in its entirety for all purposes. As another example, a suitable absorbent elastic nonwoven material may include a matrix of thermoplastic elastomeric nonwoven filaments present in an amount of about 3 to less than about 20% by weight of the material, with the matrix including a plurality of absorbent fibers and a super-absorbent material each constituting about 20-77% by weight of the material. U.S. Pat. No. 6,362,389 describes such a nonwoven material and is incorporated herein by reference in its entirety for all purposes. Absorbent elastic nonwoven materials are useful in a wide variety of personal care articles where softness and conformability, as well as absorbency and elasticity, are important.

The absorbent web may also be a nonwoven web comprising synthetic fibers. The web may include additional natural fibers and/or superabsorbent material. The web may have a density in the range of about 0.1 to about 0.45 grams per cubic centimeter. The absorbent web can alternatively be a foam.

When incorporated into an absorbent article such as the diaper 10 as shown in FIG. 1A, the electroactive display may have multiple and numerous purposes and uses. For instance, the electroactive display 12 may serve primarily for aesthetic or entertainment purposes, by displaying graphics on the front panel that depicts a scene that shifts periodically such as an animated character, a flag that waves, a fish that appears to swim, a vehicle that traverses across the article, and the like. One example of technology suitable for entertainment purposes is that of HASBRO© STAR WARS™ action figures in which RFID tags in the figures contain digitized sound clips that are read by a central station and converted to audible sound played on speakers in the central station when an animated figure is nearby. Similar concepts can be adapted for the present invention.

In other embodiments, the electroactive display 12 may serve to monitor functions and provide dynamic graphics that respond to a condition of the user or the product. For example, in infant care or child care products, a sensor may be connected to the electroactive display to indicate if there is wetness or a bowel movement. A microcircuit can electrically modify the graphics being displayed in response to a positive reading by the sensor.

For example, as shown in FIGS. 1A-1D and in FIG. 2, the electroactive display 12 is in communication with a sensor 44. In order to indicate wetness or a bowel movement, for instance, the sensor may be a moisture sensor, a conductivity sensor, or a temperature sensor. Suitable sensors that may be used in the present invention are disclosed in, for instance, U.S. Pat. No. 5,796,345, U.S. Pat. No. 6,596,918, and PCT International Publication Number WO 03/048998, which are all incorporated herein by reference. In one embodiment, the sensor may be incorporated with a power supply.

As shown in FIGS. 1A-1D and in FIG. 2, the sensor 44 is electrically connected to the electroactive display 12 and the battery 30. Although a simple circuit is shown in the drawings, it should be understood that various other circuit elements may be present, including an integrated circuit chip if desired.

Once the sensor 44 is activated due to sensing a change in the condition of the absorbent article, the sensor 44 can be configured to cause a predetermined change to the image that is being displayed by the electroactive display 12. For example, in one embodiment, the sensor may cause the electroactive display 12 to completely shutdown. In other embodiments, however, the electroactive display 12 may, for instance, turn from bright colors to a dull gray color. In other particular embodiments, for exemplary purposes, a rainbow may vanish and turn into a cloud or, raindrops may be shown falling within the image.

By having the image change in such a noticeable manner, a child wearing the diaper 10 is prone to associate the image change with the wetting of the article. In this manner, the electroactive display 12 serves not only as an indicator but also as a training tool for potty training children.

In one particular embodiment, the electroactive display may also include an internal timer that provides positive feedback to a child in a potty training program. For example, when the child has remained dry for a period of time, graphics may appear to provide encouragement. For exemplary purposes, for instance, an animated character may appear to congratulate the child.

In addition to sensors designed to indicate wetness or a bowel movement, other sensors may also be incorporated into the article of the present invention. For instance, in one embodiment, a biosensor may be incorporated into the article. The biosensor may be activated when contacted with an analyte contained in a body fluid. The analyte may be, for instance, a protein, a glycoprotein, an antibody, an antigen, hemoglobin, an enzyme, a metal salt, a hormone, and the like. Once activated, the biosensor may be configured to cause the electroactive display to display a symbol or message for indicating a particular problem.

For instance, healthcare products and incontinence products in care-giving institutions may be adapted such that the displayed graphics are modified in response to humidity, temperature or a host of bio-indicators. In incontinence articles, for example, biosensors for a variety of disease conditions (e.g., cancer, diabetes, etc.) may be present and associated with respective warning indicators that are activated when a positive reading for a target analyte occurs. In one particular embodiment, the biosensor may be configured to sense a particular protein that would indicate a kidney problem. The article of the present invention may also monitor the hydration level with a sensor quantifying the ionic strength in urine, or could monitor sugar in urine or indicators for yeast in feminine care products.

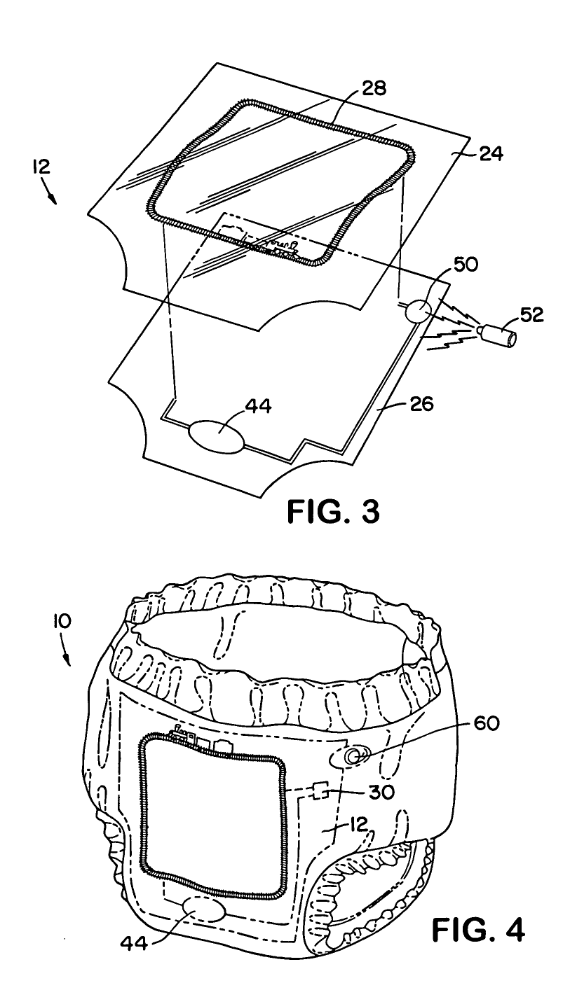

Referring to FIG. 3, another embodiment of an electroactive display 12 made in accordance with the present invention is shown. Like reference numerals have been used to indicate similar elements or features. As shown, the electroactive display 12 includes a first electrode 24, a second electrode 26, and an electrochromic composition 28 positioned between the two electrodes. A sensor 44 is also shown in communication with the electrochromic composition 28.

In this embodiment, the power supply or battery comprises a radiofrequency identification device that is capable of harnessing energy when positioned to receive radiofrequencies from a RF source 52. Radiofrequency identification devices are low-cost, passive “smart” chips or “tags” that can be easily imbedded within the electroactive display 12 or an absorbent product. The radiofrequency identification devices may convey information about the product via a scanner. The smart tags are generally small labels or the like with a miniature embedded antennae. In the past, readers or scanners have interrogated the smart tag with an electronic signal. The tag in turn generates an electromagnetic pulse response that is readable by the scanner, the response containing the product information.

When used in the process of the present invention, electromagnetic energy may be harvested by the RFID device. The RFID device may include an antennae adapted to harvest energy at a particular frequency. When a sufficiently strong RF source at the target frequency is present, as when scanning by a nearby RFID scanner is underway, the energy harvested by the antennae can modify the appearance of the electroactive display 12.

Incorporating an RFID device into the electroactive display 12 may provide various benefits and advantages. For instance, the RFID device may provide an alert to the user that scanning is being done, or may assist someone in scanning a product and verifying its location or its authenticity.

In another embodiment, the RFID device associated with the electroactive display can be used as an authentication scheme. In this embodiment, authentication of a product or document requires not only that an electronic product code be read from the RFID device, but also that a color-changing graphical display be activated when the scanning is done.

In one embodiment, when incorporated into a diaper or training pant, the electroactive display 12 may be used to provide an animated story environment for a child with graphics that change in response to proximity to identified object or other devices. The RFID device 50 may serve to identify the proximity of RFID-tagged objects causing the graphics to change.

Further, it should be understood that the RFID device 50 as shown in FIG. 3 may be used alone or in conjunction with a traditional battery, such as the electrochemical cell shown in FIGS. 1A-1D.

Referring to FIG. 4, another embodiment of a diaper 10 made in accordance with the present invention is shown. Again, like reference numerals have been used to indicate similar elements. As shown, the diaper 10 includes an electroactive display 12. The electroactive display 12 may be in communication with a battery 30 and a sensor 44.

In this embodiment, the diaper 10 further includes a user activated switch 60. The user activated switch 60 may be used to electrically connect the battery 30 to the electroactive display 12 for turning on and off the active graphics and moving features. The user activated switch 60 may be used, for instance, to preserve battery power.

In one embodiment, the battery 30 may be connected to the electroactive display 12 through the use of a timing circuit. The timing circuit may be any suitable electronic timing device. The timing circuit, for instance, may be configured to cause the image being generated by the electroactive display 12 to change in a predetermined manner after a preselected period of time. As discussed earlier, in one embodiment, the image may change in order to encourage a child who is in a potty training program.

In other embodiments, however, the timing circuit may be used to indicate how long the absorbent article has been worn or to indicate that a particular action is needed. For instance, in one particular embodiment, the electroactive display 12 may be incorporated into absorbent swimwear. The timing circuit may be used to indicate the need to reapply sunscreen after a set period of time.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. An absorbent article comprising:
an outer cover having an interior surface and an exterior surface;
an absorbent structure positioned adjacent the interior surface of the outer cover;
an electroactive display disposed on the exterior surface of the outer cover, the electroactive display comprising an electrochromic composition positioned between a first electrode and a second electrode;
a battery connected to the electroactive display for providing power to the display, the battery being disposed on the absorbent article; and
wherein, when receiving power from the battery, the electroactive display is configured to create an image containing one or more active features.

2. An absorbent article as defined in claim 1, wherein the article comprises a diaper, a training pant, an incontinence product, a medical garment, a bandage, or an absorbent swimwear.

3. An absorbent article as defined in claim 1, further comprising a sensor in communication with the battery by an electrical circuit, wherein, when the sensor is activated, the electrical circuit causes a change in the appearance of the electroactive display.

4. An absorbent article as defined in claim 3, wherein the sensor comprises a moisture sensor, a conductivity sensor, or a temperature sensor.

5. An absorbent article as defined in claim 4, wherein the sensor is configured to disconnect the battery from the electroactive display when activated.

6. An absorbent article as defined in claim 4, wherein the sensor causes the features within the image created by the electroactive display to stop moving.

7. An absorbent article as defined in claim 3, wherein the sensor comprises a biosensor.

8. An absorbent article as defined in claim 7, wherein the biosensor is activated when contacted with an analyte contained in a body fluid.

9. An absorbent article as defined in claim 8, wherein the analyte comprises a protein, a glycoprotein, an antibody, an antigen, hemoglobin, an enzyme, a metal salt, or a hormone.

10. An absorbent article as defined in claim 1, wherein the electroactive display is removably attached to the outer cover.

11. An absorbent article as defined in claim 10, wherein the electroactive display is attached to the outer cover by hook and loop fasteners.

12. An absorbent article as defined in claim 1, wherein the electroactive display further comprises multiple color graphics when receiving power from the battery.

13. An absorbent article as defined in claim 12, wherein the multiple color graphics comprise two or more visually distinct colors different than a background color.

14. An absorbent article as defined in claim 1, wherein the battery comprises a radio frequency identification device that generates power when the device receives a particular radio frequency at a suitable power level.

15. An absorbent article as defined in claim 1, wherein at least one of the active features within the image is repeatedly changing when the electroactive device is receiving a current.

16. An absorbent article as defined in claim 1, wherein the electrochromic composition that is present between the electrodes is in the form of pixels that are selectively activated.

17. An absorbent article as defined in claim 1, further comprising a user activated switch for electrically connecting the battery to the electroactive display.

18. An absorbent article as defined in claim 1, wherein the electrochromic composition comprises an electrochromic compound that undergoes a reversible electron transfer reaction resulting in a pH gradient, a pH indicator, and a conductive material for transporting ions from the electrochromic compound to the pH indicator.

19. An absorbent article as defined in claim 1, wherein the first electrode is transparent.

20. An absorbent article as defined in claim 19, wherein the first electrode comprises a film containing a metal doped metal oxide.

21. An absorbent article as defined in claim 18, wherein the first electrode comprises a film comprising indium tin oxide.

22. An absorbent article as defined in claim 1, wherein the battery comprises an electrochemical cell.

23. An absorbent article as defined in claim 22, wherein the electrochemical cell includes a zinc anode and a manganese dioxide cathode.

24. An absorbent article as defined in claim 23, wherein the anode and the cathode have been printed onto a substrate.

25. An absorbent article as defined in claim 22, wherein the electrochemical cell has a thickness of less than about 1 mm.

26. An absorbent article as defined in claim 1, wherein the electroactive display has a flexibility diameter of 2 inches or less.

27. An absorbent article as defined in claim 1, wherein the electroactive display has a flexibility diameter of 1 inch or less.

28. An absorbent article comprising:
an outer cover having an interior surface and an exterior surface;
an absorbent structure positioned adjacent the interior surface of the outer cover;
an electroactive display disposed on the exterior surface of the outer cover, the electroactive display comprising an electrochromic composition positioned between a first electrode and a second electrode, the electrochromic composition being present between the electrodes in the form of pixels that are selectively activated when the electroactive display is connected to an electrical current;
an electrochemical cell connected to the electroactive display for providing power to the display, the electrochemical cell having a thickness of less than about 3 mm;
a sensor in communication with the electrochemical cell by an electrical circuit, wherein, when the sensor is activated, the electrical circuit causes a change in the appearance of the electroactive display, the sensor comprising a moisture sensor, a conductivity sensor, or a temperature sensor; and
wherein, when receiving an electrical current, the electroactive display is configured to create an image containing multiple color graphics and active features.

29. An absorbent article as defined in claim 28, wherein the electrochemical cell has a thickness of less than about 1 mm.

30. An absorbent article as defined in claim 28, wherein the article comprises a diaper, a training pant, an incontinence product, a medical garment, a bandage, or an absorbent swimwear.

31. An absorbent article as defined in claim 28, wherein the sensor is configured to disconnect the battery from the electroactive display when activated.

32. An absorbent article as defined in claim 28, wherein the sensor causes the features within the image created by the electroactive display to stop moving.

33. An absorbent article as defined in claim 28, wherein the electroactive display is removably attached to the outer cover.

34. An absorbent article as defined in claim 28, wherein the electroactive display is attached to the outer cover by hook and loop fasteners.

35. An absorbent article as defined in claim 28, wherein the active features within the image are continuously in motion when the electroactive device is receiving a current.

36. An absorbent article as defined in claim 28, further comprising a user activated switch for electrically connecting the battery to the electroactive display.

37. An absorbent article as defined in claim 28, wherein the electrochromic composition comprises an electrochromic compound that undergoes a reversible electron transfer reaction resulting in a pH gradient, a pH indicator, and a conductive material for transporting ions from the electrochromic compound to the pH indicator.

38. An absorbent article as defined in claim 28, wherein the first electrode is transparent.

39. An absorbent article as defined in claim 38, wherein the first electrode comprises a film containing a metal doped metal oxide.

40. An absorbent article as defined in claim 39, wherein the first electrode comprises a film comprising indium tin oxide.

41. An absorbent article as defined in claim 28, wherein the electrochemical cell includes a zinc anode and a manganese dioxide cathode.

42. An absorbent article as defined in claim 28, wherein the image of the electroactive display depicts an animated and active character.

43. An absorbent article as defined in claim 28, wherein the image of the electroactive display depicts a moving vehicle.

44. An absorbent article as defined in claim 28, wherein the electroactive display has a flexibility diameter of 2 inches or less.

45. An absorbent article as defined in claim 28, wherein the electroactive display has a flexibility diameter of 1 inch or less.

46. An absorbent article comprising:
an outer cover having an interior surface and an exterior surface;
an absorbent structure positioned adjacent the interior surface of the outer cover;
an electroactive display disposed on the exterior surface of the outer cover, the electroactive display comprising an electrochromic composition positioned between a first electrode and a second electrode;
a battery connected to the electroactive display for providing power to the display, the battery being disposed on the absorbent article;
a user activated switch for electrically connecting the battery to the electroactive display, the battery being connected to the electroactive display by a timing circuit; and
wherein, when the switch is activated, a current is fed from the battery through the timing circuit and to the electroactive display, the electroactive display being configured to create an image containing active features, the timing circuit causing the image to change in a predetermined manner after a preselected period of time.

47. An absorbent article as defined in claim 46, wherein the article is a potty-training device for a child.

48. An absorbent article as defined in claim 46, wherein the absorbent article comprises absorbent swimwear, and wherein the image on the electroactive display changes in a manner to indicate to a user the need to apply sunscreen.

49. An absorbent article as defined in claim 46, wherein the image on the electroactive display is changed after the preselected period to indicate that it is time to change the absorbent article.

50. An absorbent article comprising:
an outer cover having an interior surface and an exterior surface;
an absorbent structure positioned adjacent the interior surface of the outer cover;
an electroactive display disposed on the exterior surface of the outer cover, the electroactive display comprising an electrochromic composition positioned between a first electrode and a second electrode, the electroactive display being configured to create an image containing active features when receiving an electrical current; and
a radio frequency identification device in communication with the electroactive display, the radio frequency identification device being configured to change the image on the electroactive display in a predetermined manner in response to receiving radio waves from an RF source.
